(19) United States
(12) Patent Application Publication
(10) Pub. No.: US 2025/0186608 A1
(43) Pub. Date: Jun. 12, 2025

(54) NANODIAMONDS AS DELIVERY PLATFORM FOR OXIME ANTIDOTES TO CENTRAL NERVOUS SYSTEM IN ORGANOPHOSPHATE POISONING

(71) Applicant: The Curators of the University of Missouri, Columbia, MO (US)

(72) Inventors: Vadym Mochalin, Rolla, MO (US); Yevgen Karpichev, Tallinn (EE); Denys Bondar, Tallinn (EE); Pirjo Spuul, Saue (EE); Olga Smirnova, Tallinn (EE)

(73) Assignee: Tallinn University of Technology, Tallinn (EE)

(21) Appl. No.: 18/972,432

(22) Filed: Dec. 6, 2024

Related U.S. Application Data
(60) Provisional application No. 63/608,507, filed on Dec. 11, 2023.

(51) Int. Cl. A61K 47/69; A61K 35/22; A61K 47/02

(52) U.S. Cl. CPC A61K 47/6929; A61K 35/22; A61K 47/02

(57) ABSTRACT

Detonation nanodiamond nanocarrier platforms to transport quaternary oxime antidotes into the central nervous system have been developed. The nanodiamond-based AChE reactivators contain an organophosphorus poisoning antidote (e.g., a 4-hydroximinopyridinium moiety) bound to a biocompatible linker covalently attached to the nanodiamonds. These functionalized nanodiamonds successfully cross the layer of Madin-Darby Canine Kidney (MDCK) cells, the epithelial cell surrogate BBB model, and demonstrate a measurable dose-independent in vitro reactivation capacity towards human AChE inhibited by toxic organophosphorus compounds.

### CROSS-REFERENCE TO RELATED APPLICATIONS

The present application claims the priority benefit of U.S. Provisional Patent Application Ser. No. 63/608,507, filed Dec. 11, 2023, entitled NANODIAMONDS AS DELIVERY PLATFORM FOR OXIME ANTIDOTES TO CENTRAL NERVOUS SYSTEM IN ORGANOPHOSPHATE POISONING, the entirety of which is incorporated by reference herein.

### FEDERALLY SPONSORED RESEARCH OR DEVELOPMENT

This invention was made with government support under Contract No. W911NF-18-1-0155, awarded by the Army Research Office and Contract No. 1R15EY029813-01A1, awarded by the National Eye Institute of the National Institutes of Health. The government has certain rights in the invention.

### BACKGROUND

### Field

The present disclosure relates to nanoparticles that can be used to deliver target compounds (and particularly positively charged target compounds) across the blood-brain barrier and to central neural system.

### Description of Related Art

Acts of biological and chemical terrorism use toxic agents, for example, chemical warfare agents (CWA) to cause disease or death in man, animals, and plants. Nanotechnology offers innovative and underutilized opportunities for defence against the threats of biological and chemical warfare. Although the major world powers have agreed to abandon the use of CWAs, destroy their existing stockpiles, and control precursors, the key intermediates can be relatively easily synthesized. The risk has also been enhanced by dual use of artificial-intelligence-powered drug discovery, which could be misused for designing novel toxins. Some of the most dangerous CWAs are the nerve agents classified as G-agents, V-agents, and so-called A-agents known as Novichoks, which not long ago were used in attacks in the UK. Structurally similar to this group are organophosphorus pesticides. The nerve agents inhibit serine esterases by covalently binding to a serine residue in the catalytic site of the enzyme. Inhibition of mammal and specifically human acetylcholine esterase (AChE) is the main mechanism for nerve agent toxicity resulting in the accumulation of the neurotransmitter acetylcholine in synapses, leading to nervous and respiratory failure and death within minutes of the exposure if left untreated.

Improving individual- and community-level resilience against potential attacks becomes part of sustained investment targeted at avoiding panic and reinforcing response if CWAs were used against civilians. A core component of the “frontline” response to terrorism, first responders and spontaneous volunteers, will face health and safety risks requiring up-to-date protective equipment and antidotes. Despite numerous efforts, there are still no available nerve agent antidotes that can easily cross the blood-brain barrier (BBB), effectively restore enzyme activity in the central nervous system, (CNS), and prevent brain damage, thereby improving survival rate and quality of life of the affected individuals.

Research on the effective antidotes against organophosphorus poisoning has continued for nearly seven decades since pralidoxime (2-PAM) was employed, resulting in the development of potent AChE reactivators bearing the hydroximinomethyl pyridinium fragment in their structure. Meanwhile, the efforts to improve oxime reactivation efficacy have yielded only a limited success due to the presence of the pyridinium moiety, which prevents access of the positively charged molecules to the CNS.

The current standard treatment of poisoning by toxic organophosphorus compounds usually consists of combined administration of anticholinergic drugs (atropine) and nucleophilic agents (quaternary oximes). Anticholinergic drugs block effects of the accumulated neurotransmitter acetylcholine, while the oximes reactivate AChE inhibited by the organophosphorus compounds. However, the development of a versatile molecular scaffold with high efficiency towards inhibited AChE and the ability to cross the BBB remains a challenge. There is a need for an improved delivery system of these antidotes across the BBB.

### SUMMARY

In one embodiment, the present disclosure is broadly concerned with a method of delivering a target compound across the blood brain barrier of a subject (e.g., human and/or other animal). The method comprises administering nanodiamonds covalently bonded to: (a) said target compound; (b) a linker that is covalently bonded to said target compound; or (c) a combination of (a) and (b).

In another embodiment, the disclosure provides a nanodiamond having a surface group that is covalently bonded to a positively charged molecule chosen from pralidoxime, asoxime, obidoxime, trimedoxime, methoxime, or mixtures thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides scanning electron microscope (SEM) images of pristine ND-COOH (top left) and the oxime-functionalized nanodiamonds prepared in Example 4;

FIG. 2 shows the FT-IR spectra of ND-COOH and ND-A2 (Example 5);

FIG. 3A provides ¹³C MAS NMR spectra of ND-COOH and oxime-functionalized nanodiamonds obtained by ¹H/¹³C cross polarization (CP) sequence;

FIG. 3B provides ¹³C MAS NMR spectra of ND-COOH and oxime-functionalized nanodiamonds acquired with direct excitation (without CP);

FIG. 3C provides ¹³C MAS NMR spectra of ND-COOH and oxime-functionalized nanodiamonds obtained by ¹H/¹³C CP sequence;

FIG. 3D provides ¹³C MAS NMR spectra of ND-COOH and oxime-functionalized nanodiamonds acquired with direct excitation (without CP);

FIG. 4 is a graph showing particle size distribution (represented as intensity of scattering) of nanodiamonds suspended in MilliQ water or PBS (Example 5);

FIG. 5 provides micrographs using color to show ND-A1 entry into MDCK cells;

FIG. 6 provides micrographs using color to show ND-A1 entry into HUVEC cells;

FIG. 7 shows micrographs of HUVEC cells after 24 h exposure using color to show no fluorescence observed or caused by the oxime reactivator; and

FIG. 8 shows micrographs of HUVEC cells after 24 h of ND-A1 exposure using color to show areas of ND-A1 entry.

### DETAILED DESCRIPTION

The present disclosure is concerned with novel functionalized nanodiamonds, compositions comprising those functionalized nanodiamonds, and methods of using those functionalized nanodiamonds to deliver target compounds across the blood-brain barrier, particularly in subjects in need of an antidote to organophosphorus poisoning.

The nanodiamonds used herein can be any type of nanodiamond, including those produced by detonation of carbon-containing explosives (“detonation nanodiamonds”), those produced by high pressure and temperature, those produced by laser-assisted methods, etc., with detonation nanodiamonds being particularly suitable.

In one embodiment, the starting nanodiamonds include surface functionalization via covalent bonding, with carboxylic acid groups (—COOH groups), acyl halide groups (—COCl, —COF, —COBr, —COI), and/or amino groups (—NH₂) being particularly preferred for surface functionalization. For example, in some embodiments, about 1% to about 25%, preferably about 5% to about 25%, more preferably about 10% to about 25%, and even more preferably about 15% to about 20% of the surface carbon atoms of the nanodiamond particles bear one or more types of the above functional groups. Additionally or alternatively, the nanodiamonds comprises about 1 to about 2 functional groups per nm² of nanodiamond surface area, preferably about 1 to about 1.7 functional groups per nm² of nanodiamond surface area, and more preferably about 1.3 to about 1.5 functional groups per nm² of nanodiamond surface area.

Nanodiamonds for use herein typically comprise primary particles (i.e., individual particles) having an average particle size of about 2 nm to about 10 nm, preferably about 2 nm to about 8 nm, and more preferably about 3 nm to about 6 nm. Preferably, the nanodiamonds are of high purity (purified by air oxidation, ozone, or other techniques, see e.g., Kume et al., Sonication-assisted Hydrolysis of Ozone Oxidized Detonation Nanodiamond, Diamond and Related Materials, Vol. 103 (2020), 107705, incorporated by reference herein) and free of non-diamond carbon (graphitic, amorphous, etc.) and polymeric or other coatings.

The starting nanodiamonds are functionalized with a target compound that is to be delivered across the blood-brain barrier. Preferred target compounds include oximes, and particularly oximes that are useful in treating organophosphorus poisoning. Advantageously, the target compound also includes charged molecules, such as quaternary oximes. Examples of suitable target compounds include pralidoxime (2-PAM), asoxime (HI-6), obidoxime, trimedoxime (TMB-4), methoxime, or mixtures thereof.

The target compound is preferably bound to the nanodiamond via a linker or spacer group. The linker should be biocompatible, with suitable linkers including diamines, with preferred diamines comprising short chain poly (oxyethylene) diamines (PEG-diamines). In preferred embodiments, the diamine linker is attached covalently to the nanodiamond through one amino group and forms an amide bond via another amino group reacted with a haloacyl halide (e.g., chloride or bromide), which is bound to the target compound (e.g., corresponding pyridinium oxime) covalently. An exemplary linker comprises

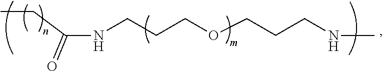

where m is 1 to 5, and n is 1 to 5.

The methods of making the functionalized nanodiamonds are shown in detail in the Examples. Broadly, the PEG-diamine linker is first activated with a protecting group (e.g., tert-butyloxycarbonyl protecting group, BOC) to form a protected linker. In some embodiments, the protected linker is then reacted with an acyl halide (preferably C₁ to C₅) to form the activated linker. Next, the desired target compound (e.g., oxime) is then grafted to the linker to form a functionalized linker, followed by deprotection of the linker (i.e., removal of the protecting group) to form the deprotected linker. Finally, the deprotected linker is grafted to the nanodiamond surface. In preferred embodiments, this grafting involves the reaction of the deprotected linker with one or more of the nanodiamond surface groups (e.g., —COOH), so that the deprotected linker is covalently bonded with that nanodiamond surface group.

The final nanodiamonds functionalized with the target compound are preferably do not include any sort of coating (i.e., no polymer, silica, metals, and/or other coating) and in the form of single particles. However, the final nanodiamonds may also form aggregates (clusters of primary particles) that typically have an average hydrodynamic diameter of about 150 nm to about 620 nm, and preferably about 250 nm to about 550 nm. In some embodiments, about 1% to about 25%, preferably about 5% to about 25%, more preferably about 10% to about 25%, and even more preferably about 15% to about 20% of the surface carbon atoms of the nanodiamond particles include one or more types of the previously described target compounds (bound via a linker, as discussed previously).

The formed functionalized nanodiamonds can then be administered to a subject (e.g., human or other animal) who has been exposed to organophosphorus compounds, such as nerve agents, toxins, pesticides, simulants for pesticides, herbicide, or mixtures thereof. Examples of the foregoing include those chosen from triphenyl phosphate, methyl paraoxon, paraoxon (POX), parathion, chlorpyrifos, chlorpyrifos oxon, malaoxon, O,O-diethyl O-(4-nitrophenyl) phosphorothioate (parathion), O,O-Dimethyl O-(3-methyl-4-nitrophenyl) phosphorothioate (fenitrothion), isopropyl methylphosphonofluoridate (GB or Sarin), (RS)-ethyl N,N-dimethylphosphoramidocyanidate (GA or Tabun), cyclohexyl methylphosphonofluoridate (GF or cyclosarin), (O-ethyl-S-[2(diisopropylamino)ethyl]methylphosphonothioate) (VX), O-butyl-S-[2-(diethylamino)ethyl]methylphosphonothioate (CVX), S-[2-(diethylamino)ethyl]-O-(2-methylpropyl) methylphosphonothioate (VR), 3,3-dimethylbutan-2-yl methylphosphonofluoridate (GD or Soman), 2-(dimethylamino)ethyl N,N-dimethylphosphoramidofluoridate (GV), methyl-(1-(diethylamino)ethylidene)phosphonamidofluoridate (A-230), methoxy-(1-(diethylamino)ethylidene)phosphoramidofluoridate (A-232), ethyl N-[(1E)-1-(diethylamino)ethylidene]-phosphoramidofluoridate (A-234), or mixtures thereof.

Administration can be carried out by providing a therapeutically effect amount via any typical method, including intravenous injection, parenteral administration, intranasal administration, oral administration, and/or most other methods of administration. The functionalized nanodiamonds can be part of a composition that includes carriers and other ingredients conventionally used for delivering such treatments. In some embodiments, the functionalized nanodiamonds are the only active ingredient in the formulation. That is, in these embodiments, the formulation consists essentially of, or even consists of, the functionalized nanodiamonds, carrier, and optionally any adjuvants.

In one or more embodiments, the formulation is essentially free of polymers. That is, the formulation comprises less than about 2% by weight, preferably less than about 1% by weight, more preferably less than about 0.5% by weight, and most preferably about 0% by weight polymers, based on the total weight of the formulation taken as 100% by weight.

In one or more embodiments, the formulation is essentially free of nanoparticles other than the functionalized nanodiamonds. That is, the formulation comprises less than about 2% by weight, preferably less than about 1% by weight, more preferably less than about 0.5% by weight, and most preferably about 0% by weight nanoparticles other than the functionalized nanodiamonds, based on the total weight of the formulation taken as 100% by weight.

In some embodiments, the formulation is essentially free of proteins (e.g., albumin). That is, the formulation comprises less than about 2% by weight, preferably less than about 1% by weight, more preferably less than about 0.5% by weight, and most preferably about 0% by weight total protein, based on the total weight of the formulation taken as 100% by weight.

In some embodiments, the formulation is essentially free of metal-organic frameworks (MOFs). That is, the formulation comprises less than about 2% by weight, preferably less than about 1% by weight, more preferably less than about 0.5% by weight, and most preferably about 0% by weight MOFs, based on the total weight of the formulation taken as 100% by weight.

Advantageously, the functionalized nanodiamonds at a concentration of about 10 μg/mL to about 100 μg/mL have an apparent permeability coefficient according to the MDCK blood-brain barrier model (determined as described in Example 6) of 3×10⁻⁶ cm/s or greater, preferably about 4×10⁻⁶ cm/s or greater, more preferably about 8×10⁻⁶ cm/s or greater, and even more preferably about 10×10⁻⁶ cm/s or greater.

Another advantage of the invention disclosed herein is that the functionalized nanodiamonds (and therefore the target compound with which the nanodiamond is functionalized) can be internalized by the target cells. That is, in one or more embodiments, MDCK cells treated with these functionalized nanodiamonds at a concentration of about 50 μg/mL internalize those functionalized nanodiamonds about 24 hours after start of the treatment as visualized by confocal fluorescence microscopy at a wavelength of about 254 nm. As used herein, internalization is determined as described in Example 6.

In some embodiments, MDCK cells treated with these functionalized nanodiamonds at a concentration of about 100 μg/mL internalize those functionalized nanodiamonds about 3 hours after start of the treatment as visualized by confocal fluorescence microscopy at a wavelength of about 254 nm.

In one or more embodiments, HUVEC cells treated with these functionalized nanodiamonds at a concentration of about 10 μg/mL internalize those functionalized nanodiamonds about 1 hour after start of the treatment as visualized by confocal fluorescence microscopy at a wavelength of about 254 nm.

The present invention also provides the advantage of reactivating AChE inhibited by toxic organophosphorus compounds. That is, at a concentration of about 10 μg/mL, the functionalized nanodiamonds described herein have an AChE reactivation potency R of about 0.55% or greater, preferably about 0.85% or greater, and more preferably about 1% greater, against isopropyl methylphosphonofluoridate (GB). As used herein “AChE reactivation potency R” is determined as described in Example 6.

Additionally or alternatively, at a concentration of about 10 μg/mL, the functionalized nanodiamonds described herein have an AChE reactivation potency R of about 0.7% or greater, preferably about 0.75% or greater, and more preferably about 0.8% or greater, against O-ethyl S-diisopropylaminomethyl methylphosphonothiolate (VX).

Additionally or alternatively, at a concentration of about 10 μg/mL, the functionalized nanodiamonds described herein have an AChE reactivation potency R of about 0.95% or greater, preferably about 1.8% or greater, and more preferably about 2.4% or greater, against O,O-diethyl O-(4-nitrophenyl) phosphate (POX).

Additional advantages of the various embodiments will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present disclosure encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the disclosure. It is to be understood, however, that these examples are provided by way of illustration, and nothing therein should be taken as a limitation upon the overall scope.

Broadly, in the following Examples, the short-chain polyethylene glycol (PEG) was utilized as an example spacer connecting nanodiamond surfaces with the reactivating moiety. 4-Hydroximinopyridine was grafted to the linker, resulting in quaternary oximes on the nanoparticle surfaces. The general outline of this synthesis process is shown in Scheme A, which is referenced in the following Examples.

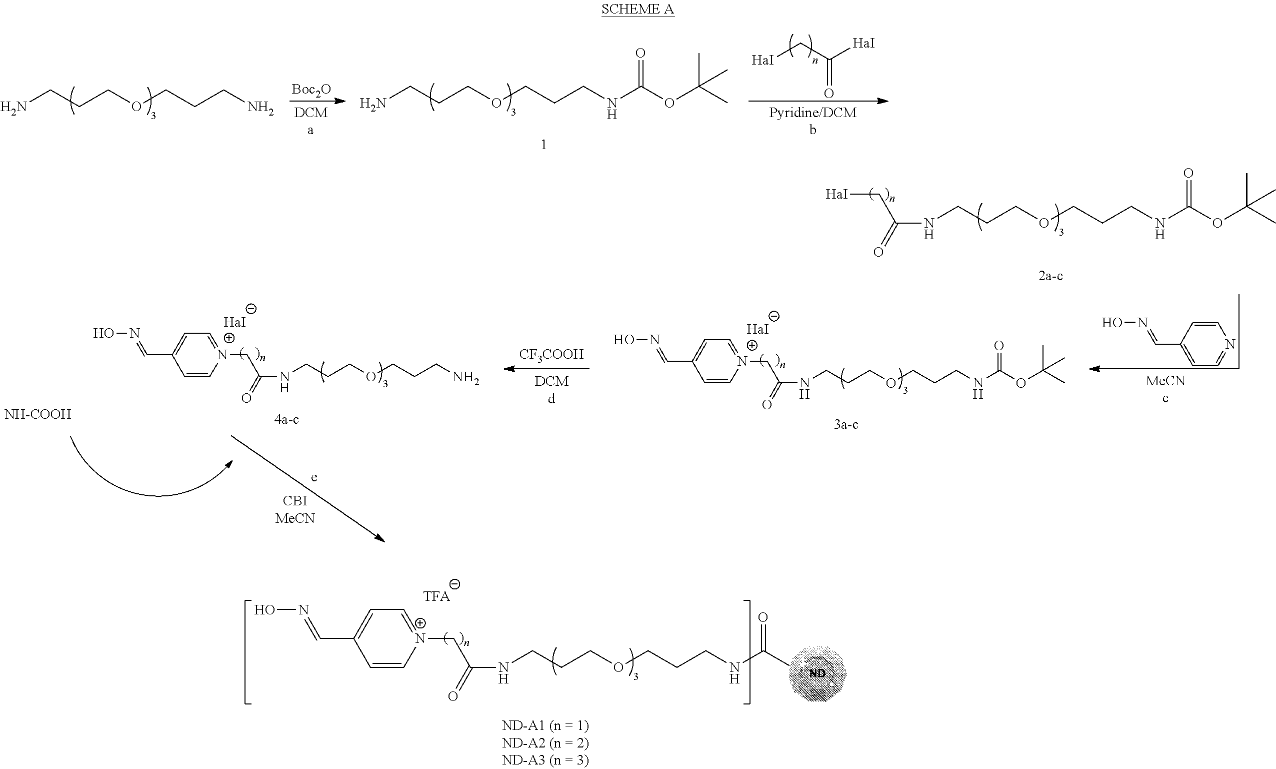

### Materials and Methods

### 1. Reagents and Chemicals

All chemicals and solvents were purchased from Sigma-Aldrich, Alfa Aesar, Fisher Scientific, or TCI Europe and used without further purification. Deionized water from a Milli-Q system was used in all sample preparations. Pralidoxime chloride was prepared in the Department of Toxicology and Military Pharmacy, Military Faculty of Medicine, University of Defense (Czech Republic). Octadecylsilane-bonded silica gel SPE column (Phenomenex) was purchased from Chromservis s.r.o. (Czech Republic). GB (isopropyl methylphosphonofluoridate) and VX (O-ethyl S-diisopropylaminomethyl methylphosphonothiolate) with purity >95% were obtained from the Military Technical Institute in Brno, Czech Republic. Paraoxon (O,O-diethyl O-(4-nitrophenyl) phosphate, POX) of analytical grade was purchased from Sigma-Aldrich.

Nanodiamond powder UD90 produced by detonation synthesis was supplied by NanoBlox, Inc., USA. The powder was characterized and purified from non-diamond carbon by a well-known standard procedure: oxidation in air followed by reflux in ˜35 wt % aqueous HCl to remove traces of metals and metal oxides. After the HCl reflux, the ND was allowed to settle to the bottom of the flask. The excess HCl with dissolved salt was removed. ND powder was separated by centrifugation, rinsed multiple times with deionized water until neutral pH, and then dried in an oven at 110° C. overnight. The purified ND with acidic groups on the surface, labeled ND-COOH, was a starting material for all subsequent functionalizations in this work.

### 2. Characterization of Products

As an additional tool to confirm conversion, ion exchange of trifluoroacetic acid (TFA) counterion in the final products was monitored by ¹⁹F NMR following the procedure developed by Okaru, A. O. et al., Application of ¹⁹F NMR spectroscopy for content determination of fluorinated pharmaceuticals, J. Anal. Methods Chem. 2017, (2017), incorporated by reference herein. The High-Resolution Mass Spectroscopy (HRMS) identification of compounds was performed with an Agilent 6540 UHD Accurate-Mass Q-TOF LC/MS G6540A mass spectrometer. IR spectra were collected with a Tensor 27 FT-IR spectrometer. NMR spectra were recorded with Bruker Avance III 400 MHz NMR spectrometer operated at 400 MHz for ¹H NMR and 101 MHz for ¹³C NMR. Spectra were recorded in deuterated chloroform (CDCL₃) or dimethyl sulfoxide (DMSO-d₆) where appropriate. All chemical shifts δ, ppm are relative to the internal standard TMS; coupling constants J are measured in Hertz (Hz). ¹³C MAS NMR spectra were recorded with a Bruker AVANCE-II spectrometer at 14.1 T magnetic field using a home-built MAS probe for 25×4 mm Si₃N₄ rotors, spinning at frequency 12.5 kHz. The spectra were recorded with a simple 90-degree pulse (4.5 μs) excitation followed by acquisition in high field (H₁=100 kHz) of proton decoupling. The relaxation delay between the excitations was 10 s. All intensities are normalized to the number of accumulations and to the weight of the sample to allow for quantitative estimates of the ratios of different carbon sites. SEM imaging was performed using Zeiss Ultra 55 with in-lens secondary electron detection at a 4 kV accelerating voltage.

### 3. Dynamic Light Scattering (DLS)

The hydrodynamic diameter, particle size distribution, and zeta potential of the ND aggregates suspended in water and PBS were measured using ZetaSizer Nano S (Malvern Instruments) with He-Ne laser (633 nm, 10 mW) in back-scattering geometry (θ=173°). The data obtained was processed using Malvern DTS Software 7.11. Before the measurements, the samples were sonicated in TORBEO® 36810 Ultrasonic Cell Disruptor for 90 s (2×45 s) at 8 W effective radiated power for sonication.

### 4. MDCK Assay Double-well Experiment

The Madin-Darby Canine Kidney (MDCK) assay was used to evaluate the passage of nanodiamonds and produced nanodiamond conjugates through the MDCK cell membrane. (Gorecki, L. et al. Structure-activity relationships of dually-acting acetylcholinesterase inhibitors derived from tacrine on N-methyl-D-Aspartate receptors, Eur. J. Med. Chem. 219, 113434 (2021), incorporated by reference herein). The cells were seeded on a polycarbonate membrane (1.12 cm² area with 3 μm pores) of the 12-well plates with 12 mm inserts. The apparent permeability coefficient (Pₐₚₚ) was calculated from Equation (1):

$${P}_{app} = ( \frac{dC}{dt} ) \cdot \frac{{V}_{r}}{( A \cdot {C}_{0} )} \qquad ( 1 )$$

A—area of the well/cell monolayer; dC/dt—rate of permeation; Vᵣ—volume of the receiving compartment; and C₀-initial concentration of a tested compound. The tightness of MDCK monolayer was assessed by the permeability of fluorescein isothiocyanate (FITC) in concentration 0.4 mg/mL. Monolayer integrity was confirmed after each experiment, by ensuring that FITC level in the acceptor compartment did not surpass 1% of its initial concentration in the donor compartment after 6 hours.

### 5. HUVEC and MDCK Assays for Cell Internalization Studies

Pooled human umbilical vein endothelial cells (HUVEC) (200P-05N) were cultured in Endothelial Cell Growth Medium (211-500), both from Cell Applications, Inc.; passages between 3 and 6 were used. 7.5×10⁴ cells were plated for each experiment onto gelatin-coated coverslips (0.2% gelatin, G1393-20ML, Sigma). Astrocyte conditioned medium (1811, ScienCell Research Laboratories, Inc.) mixed 50:50 with Endothelial Cell Growth Medium was used to stimulate cell-cell junction formation. MDCK cells (84121903-1VL, Sigma-Aldrich) were cultured in Minimum Essential Medium (MEM) (15-010-CV, Corning®) supplemented with 10% fetal bovine serum (FBS, Merck®). After forming confluent cell layers, NDs were added in 10, 50, or 100 μg/mL concentrations. Cells were incubated at 37° C., 5% CO₂ for 1, 3 or 24 h, followed by washing with PBS and fixation. CytoSMART cell counter (Corning™) was used for cell counting.

Fixation of cells was performed at indicated time-points in 4% paraformaldehyde for 30 min at room temperature followed by permeabilization with 0.1% Triton®X-100 (AppliChem GmbH) for 2 min and incubation in blocking solution for 5 min at RT (1% BSA and 2% FBS in PBS). Primary and secondary antibody dilutions were made in blocking solution. Cell-cell junctions (TJs) were stained with ZO-1 (33-910, Mouse Monoclonal Antibody, ZO1-1A12; Thermo Scientific™) followed by Goat anti-Mouse IgG Alexa Fluor®567 (A-11004, Invitrogen™); F-actin was stained with Alexa Fluor 546-phalloidin (Invitrogen) and nuclei were visualized with Hoechst 33342 (62249, Thermo Scientific™). Cells were mounted using ProLong® Gold Anti-fade Reagent (Life Technologies).

### 6. Imaging and Image Analysis

The functionalized nanodiamonds ND-A1, ND-A2, and ND-A3 show bright green fluorescence when irradiated with ultraviolet light with a wavelength of 254 nm and, in contrast to ND-COOH, can be successfully imaged by means of fluorescent and confocal microscopy. ND-treated stained cells were analyzed with a confocal microscope Zeiss LSM 510 DUO using 63×/1.4 oil immersion objective and point scanning Ar lasers at wavelengths 488, 561 and 405 nm. Optical sections were acquired using sequential unidirectional scanning. Images were analyzed with Fiji ImageJ program (Schindelin, J. et al. Fiji: an open-source platform for biological-image analysis, Nat. Methods. 9, 676-682 (2012), incorporated by reference herein.) and Bitplane Imaris (Oxford Instruments) was used for 3D rendering and section view.

### 7. AChE Reactivation Assay

Reactivation potency of the new oximes and standard reactivator 2-PAM was evaluated using human recombinant AChE. The enzyme was inhibited by GB, VX and paraoxon (POX) in propan-2-ol (0.01 mM; 60 min). The excess of inhibitor was removed using octadecylsilane-bonded silica gel SPE cartridge. The inhibited AChE was incubated for 10 min with a reactivator solution (suspension) at 37° C. Then the reaction was initiated by adding acetylthiocholine and AChE activity was measured spectrophotometrically at 412 nm, following the modified method of Ellman, G. L., Courtney, K. D., Andres, V. & Featherstone, R. M., A new and rapid colorimetric determination of acetylcholinesterase activity, Biochem. Pharmacol. 7, 88-95 (1961), incorporated by reference herein, as detailed previously (Jun, D., Musilova, L., Musilek, K. & Kuca, K. In vitro ability of currently available oximes to reactivate organophosphate pesticide-inhibited human acetylcholinesterase and butyrylcholinesterase, Int. J. Mol. Sci. 12, 2077-2087 (2011); Spilovska, K. et al. Novel tacrine-scutellarin hybrids as multipotent anti-A1zheimer's agents: design, synthesis and biological evaluation, Molecules 22, 1006 (2017), each incorporated by reference herein.). Each concentration of reactivator was assayed in triplicate. The data from these assays were used to calculate reactivation potency R (Equation 2), corrected for oximolysis and inhibition of AChE by the reactivator.

$$R = ( 1 - \frac{Δ {A}_{0} - Δ {A}_{r}}{Δ {A}_{0} - Δ {A}_{i}} ) \times 100 [ \% ] \qquad ( 2 )$$

R—reactivation potency; ΔA₀—absorbance change by intact AChE (phosphate buffer); and ΔAᵢ—absorbance change by AChE after exposure.

### 8. In Vitro Cell Viability Assessment

The standard MTT assay (Sigma-Aldrich) was used according to the manufacturer's protocol on the CHO-K1 cells (Chinese hamster ovary, ECACC, Salisbury, UK). The cells were cultured according to ECACC recommended conditions and seeded at a density 8000 per well as described by Spilovska, K. et al., Novel tacrine-scutellarin hybrids as multipotent anti-Alzheimer's agents: design, synthesis and biological evaluation, Molecules 22, 1006 (2017), incorporated by reference herein. The tested compounds were dissolved (or dispersed in case of nanocarriers) in the F-12 growth medium. Cells were exposed to a tested compound for 24 hours. Then the medium was replaced by a medium containing 10 μM of MTT and the cells were allowed to produce formazan for 3 h under surveillance. Thereafter, the medium with MTT was removed, crystals of formazan were dissolved in DMSO (100 μL), and cell viability was assessed by the amount of formazan produced, which was determined spectroscopically. Absorbance was measured at 570 nm with 650 nm reference wavelength on Synergy HT (BioTek, USA). Half-maximal inhibitory concentration (IC₅₀) was calculated from the control-subtracted triplicates using four-parameter non-linear regression carried out in GraphPad Prism 5 software.

### Example 1

### Preparation of Linker

### 1. Preparation of tert-Butyl 3-(2-(2-(3-aminopropoxy)ethoxy)ethoxy)propylcarbamate (1 in Scheme A)

In the reaction designated by “a” in Scheme A, di-tert-butyl dicarbonate (2.2 g, 2 mmol; “BOC₂O” in Scheme A) in CH₂Cl₂ (15 mL) was added dropwise to a solution of 4,7,10-trioxa-1,13-tridecanediamine (8.8 g, 40 mmol) in CH₂Cl₂ (100 mL; “DCM” in Scheme A) at 0° C. and stirred for 12 hours. The reaction was checked by TLC using a solution of ninhydrin as a TLC stain and stopped by adding MeOH (5 mL). The reaction mixture was then concentrated and purified by column chromatography to obtain the product 1 (2.4 g, 7 mmol, 75%) as a colorless oil. R_{f}=0.3 (MeOH/CH₂Cl₂1:10).

HRMS (EI, m/z): calcd. for C₁₅H₃₂N₂O₅ [M+H]⁺321.2384, found 321.2392.

¹H NMR (400 MHz, CDCL₃): δ 5.11 (s, 1H), 3.65-3.51 (m, 12H), 3.22 (q, 2H, J=6.3), 2.78 (t, 2H, J=6.7), 1.80-1.65 (m, 4H), 1.43 (s, 9H), 1.27 (s, 2H).

¹³C NMR (101 MHz, CDCL₃): δ 156.20, 78.98, 70.77, 70.73, 70.38, 70.34, 69.74, 69.60, 39.77, 38.67, 33.57, 29.74, 28.59.

### 2. Preparation of tert-Butyl-(1-bromo-2-oxo-7,10,13-trioxa-3-azahexadecan-16-yl)carbamate (2a in Scheme A)

In a first version of the reaction designated by “b” in Scheme A, pyridine (0.805 mL, 10 mmol) was added to a solution of 2.4 g (7 mmol) of the tert-butyl 3-(2-(2-(3-aminopropoxy) ethoxy)ethoxy)propylcarbamate prepared in Part 1 of this Example 1 in CH₂Cl₂ (40 mL) and stirred for 30 min. A 2-bromoacetyl bromide (0.87 mL, 10 mmol) was then added dropwise in an ice bath. After 30 minutes, the ice bath was removed, and the reaction was allowed to stir at room temperature for 12 hours under a nitrogen atmosphere. The reaction mixture was checked by TLC and concentrated under reduced pressure. Then, the mixture was extracted with 2M HCl (15 mL) and CH₂Cl₂ (25 mL) three times. The combined organic solution was dried over MgSO4 and concentrated in a vacuum to obtain the product 2a (2.46 g, 5.6 mmol, 79%) as a brown liquid. R_{f}=0.36 (MeOH/CH₂Cl₂ 1:10).

HRMS (EI, m/z): calcd. for C₁₇H₃₃BrN₂O₆ [M+H]⁺441.1595, found 441.1601.

¹H NMR (400 MHz, CDCL₃): δ 7.21 (bs, 1H), 4.94 (bs, 1H), 3.85 (s, 2H), 3.68-3.56 (m, 10H), 3.53 (t, 2H, J=6.0), 3.41 (q, 2H, J=5.9), 3.21 (q, 2H, J=6.3), 1.86-1.71 (m, 4H), 1.43 (s, 9H).

¹³C NMR (101 MHz, CDCL₃): δ 165.69, 156.19, 79.11, 70.68, 70.67, 70.66, 70.49, 70.34, 69.68, 39.13, 38.62, 29.80, 29.41, 28.63, 28.58.

### 3. Preparation of tert-Butyl (17-chloro-15-oxo-4,7,10-trioxa-14-azaheptadecyl)carbamate (2b in Scheme A)

In a second version of the reaction designated by “b” in Scheme A, pyridine (0.805 mL, 10 mmol) was added to a solution of 2.4 g (7 mmol) of the tert-butyl 3-(2-(2-(3-aminopropoxy)ethoxy)ethoxy) propylcarbamate prepared in Part 1 of this Example 1 in CH₂Cl₂ (40 mL) and stirred for 30 min. Then, 3-chloropropanoyl chloride (0.95 mL, 10 mmol) was added dropwise in an ice bath. After 30 minutes, the ice bath was removed, and the reaction was allowed to stir at room temperature for 24 hours under a nitrogen atmosphere. The reaction mixture was checked by TLC and concentrated under reduced pressure. Then, the mixture was extracted with 2 M HCl and CH₂Cl₂ three times. The combined organic solution was dried over MgSO4 and concentrated in a vacuum to obtain the product 2b (2.14 g, 5.2 mmol, 74%) as a yellow liquid. R_{f}=0.33 (MeOH/CH₂Cl₂ 1:10).

HRMS (EI, m/z): calcd. For C₁₈H₃₅ClN₂O₆ [M+H]⁺411.2256, found 411.2253.

¹H NMR (400 MHz, CDCL₃): δ 6.61 (bs, 1H), 4.94 (bs, 1H), 3.79 (t, 2H, J=6.6), 3.68-3.49 (m, 12H), 3.38 (q, 2H, J=5.9), 3.20 (q, 2H, J=6.4), 2.60 (t, 2H, J=6.7), 1.83-1.71 (m, 4H), 1.42 (d, 9H, J=2.1).

¹³C NMR (101 MHz, CDCL₃): δ 169.48, 156.19, 79.13, 70.62, 70.59, 70.32, 70.26, 70.15, 69.61, 40.46, 39.70, 38.59, 38.34, 29.81, 28.74, 28.57.

### 4. Preparation of tert-Butyl (18-chloro-15-oxo-4,7,10-trioxa-14-azaoctadecyl)carbamate (2c in Scheme A)

In a third version of the reaction designated by “b” in Scheme A, pyridine (0.805 mL, 10 mmol) was added to a solution of 2.24 g (7 mmol) of the tert-butyl 3-(2-(2-(3-aminopropoxy)ethoxy)ethoxy) propylcarbamate prepared in Part 1 of this Example 1 in CH₂Cl₂ (40 mL) and stirred for 30 min. Then a 4-chlorobutanoyl chloride (1.37 mL, 10 mmol) was added dropwise in an ice bath. After 30 minutes, the ice bath was removed, and the reaction was allowed to stir at room temperature for 24 hours under a nitrogen atmosphere. The reaction mixture was checked by TLC and concentrated under reduced pressure. Then, the mixture was extracted with 2 M HCl and CH₂Cl₂ three times. The combined organic solution was dried over MgSO₄ and concentrated in a vacuum to obtain the product 2c (1.84 g, 4.34 mmol, 62%) as a yellow liquid. R_{f}=0.31 (MeOH/CH₂Cl₂ 1:10).

HRMS (EI, m/z): calcd. for C₁₉H₃₇ClN₂O₆ [M+H]⁺425.2413, found 425.2417.

¹H NMR (400 MHz, CDCL₃): δ 6.42 (bs, 1H), 4.96 (bs, 1H), 3.63-3.54 (m, 12H), 3.50 (t, 2H, J=6.0), 3.34 (q, 2H, J=5.9), 3.19 (q, 2H, J=6.4), 2.31 (t, 2H, J=7.2 Hz), 2.11-2.04 (m, 2H), 1.79-1.68 (m, 4H), 1.41 (s, 9H).

¹³C NMR (101 MHz, CDCL₃): δ 171.71, 156.14, 79.02, 70.60, 70.58, 70.54, 70.30, 70.20, 69.63, 44.72, 38.59, 38.23, 33.33, 29.75, 28.91, 28.52, 28.32.

### Example 2

### Grafting of 4-hydroximinopyridinium Fragment on Linker

In the reaction designated by “c” in Scheme A, pyridine-4-carboxaldoxime (610 mg, 5 mmol) was added to a solution of the corresponding N-haloacyl derivative 2a, 2b, or 2c (5 mmol) of Parts 2-4 of Example 1 in dry acetonitrile (40 mL) and stirred for 30 min. The reaction mixture was refluxed for 12 hours, then checked by TLC, and finally concentrated under reduced pressure to obtain the product 3a, 3b, or 3c, respectively, as a brown liquid. Yields were over 90%.

### 1. (E)-1-(2,2-Dimethyl-4,20-dioxo-3,9,12,15-tetraoxa-5,19-diazahenicosan-21-yl)-4-((hydroxy-imino)methyl)pyridin-1-ium bromide 3a

¹H NMR (400 MHz, DMSO-d₆): δ 12.87 (s, 1H), 8.94 (d, 2H, J=6.8), 8.70 (bs, 1H), 8.44 (s, 1H), 8.26 (d, 2H, J=6.9), 6.75 (bs, 1H), 5.44 (s, 2H), 3.50-3.42 (m, 8H), 3.37-3.33 (m, 4H), 3.17 (q, 2H, J=6.7), 2.94 (q, 2H, J=6.6), 1.68 (p, 2H, J=6.6), 1.57 (t, 2H, J=6.7), 1.35 (s, 9H).

¹³C NMR (101 MHz, DMSO-d₆): δ 164.20, 155.58, 148.84, 146.42, 145.17, 123.43, 79.32, 77.41, 69.76, 69.62, 69.54, 68.08, 67.80, 61.03, 37.21, 36.37, 29.72, 29.06, 28.27.

### 2. (E)-1-(2,2-Dimethyl-4,20-dioxo-3,9,12,15-tetraoxa-5,19-diazadocosan-22-yl)-4-((hydroxy -imino)methyl)pyridin-1-ium chloride 3b

¹H NMR (400 MHz, DMSO-d₆): δ 11.82 (s, 1H), 8.58 (d, 2H, J=6.0), 8.16 (s, 1H), 7.96 (bs, 1H), 7.53 (d, 2H, J=6.1), 6.73 (bs, 1H), 3.77 (t, 2H, J=6.4), 3.51-3.44 (m, 8H), 3.41-3.35 (m, 4H), 3.11 (q, 2H, J=6.5), 2.96 (q, 2H, J=6.6), 2.54 (t, 2H, J=6.4), 1.60 (dq, 4H, J=13.3, 6.6), 1.36 (s, 9H).

¹³C NMR (101 MHz, DMSO-d₆): δ 168.76, 155.61, 150.25, 150.12, 146.63, 140.38, 120.62, 77.40, 69.81, 69.58, 68.14, 68.01, 41.14, 38.35, 37.27, 37.20, 35.87, 29.76, 29.32, 28.26.

### 3. (E)-1-(2,2-Dimethyl-4,20-dioxo-3,9,12,15-tetraoxa-5,19-diazatricosan-23-yl)-4-((hydroxyimino)methyl)pyridin-1-ium chloride 3c

¹H NMR (400 MHz, DMSO-d₆): δ 13.04 (s, 1H), 9.14 (d, 2H, J=6.6), 8.44 (s, 1H), 8.22 (d, 2H, J=6.8), 8.19 (bs, 1H), 6.77 (bs, 1H), 4.63 (t, 2H, J=6.6), 3.48-3.43 (m, 8H), 3.35 (td, 4H, J=6.4, 3.0), 3.03-2.96 (m, 2H), 2.92 (q, 2H, J=6.8), 2.17 (p, 4H, J=6.4), 1.56 (p, 4H, J=6.5 Hz), 1.34 (s, 9H).

¹³C NMR (101 MHz, DMSO-d₆): δ 170.65, 155.61, 148.51, 145.61, 145.24, 144.89, 123.92, 77.40, 69.78, 69.70, 69.56, 68.12, 59.91, 37.24, 35.83, 31.46, 29.74, 29.29, 28.29, 27.10, 26.68.

### Example 3

### Removal of Protective Group From Functionalized Linker

In the reaction designated by “d” in Scheme A, the corresponding Boc-protected 4-PAM construct 3a, 3b, or 3c (4 mmol) prepared in Example 2 was individually dissolved in dry dichloromethane (CH₂Cl₂) (40 mL). Trifluoroacetic acid (3.1 mL, 40 mmol) was added, and the solution was stirred under a nitrogen atmosphere. The reaction mixture was checked by thin layer chromatography (TLC) and concentrated under reduced pressure to obtain the product 4a, 4b, or 4c, respectively as a viscous oil with quantitative yield.

### 1. (E)-1-(16-Ammonio-2-oxo-7,10,13-trioxa-3-azahexadecyl)-4-((hydroxyimino)methyl)pyridin-1-ium-2-ylium 4a

¹H NMR (400 MHz, DMSO-d₆): δ 13.01 (s, 1H), 8.92 (d, 2H, J=6.7), 8.72 (bs, 1H), 8.43 (s, 1H), 8.25 (d, 2H, J=6.8), 7.88 (bs, 3H), 5.41 (s, 2H), 3.51 (m, 4H), 3.45 (m, 8H), 3.18 (q, 2H, J=6.6), 2.86-2.82 (m, 2H), 1.80-1.76 (m, 2H), 1.68 (p, 2H, J=6.6).

¹³C NMR (101 MHz, DMSO-d₆): δ 164.30, 158.60, 158.29, 148.96, 146.48, 145.21, 123.49, 121.70, 118.72, 115.75, 69.79, 69.71, 69.69, 69.58, 69.52, 67.84, 67.39, 61.09, 36.76, 36.46, 29.12, 27.21.

### 2. (E)-1-(1-Ammonio-15-oxo-4,7,10-trioxa-14-azaheptadecan-17-yl)-4-((hydroxyimino) methyl)-pyridin-1-ium 4b

¹H NMR (400 MHz, DMSO-d₆): δ 12.45 (s, 1H), 8.78 (d, 2H, J=5.7), 8.29 (s, 1H), 8.10 (s, 1H), 8.02 (s, 3H), 7.91 (d, 2H, J=5.8), 3.75 (t, 2H, J=6.3), 3.50-3.44 (m, 10H), 3.39-3.35 (m, 2H), 3.08 (dd, 2H, J=9.9, 6.1), 2.86-2.81 (m, 2H), 2.54 (t, 2H, J=6.3), 1.81-1.76 (m, 2H), 1.61 (t, 2H, J=6.7).

¹³C NMR (101 MHz, DMSO-d₆): δ 171.14, 169.08, 159.61, 159.27, 158.94, 158.61, 145.95, 145.53, 131.99, 124.93, 122.46, 121.20, 118.25, 115.32, 79.36, 69.91, 69.82, 69.68, 69.62, 68.26, 68.14, 67.54, 57.85, 41.23, 38.46, 36.93, 36.04, 29.42, 27.30.

### 3. (E)-1-(1-Ammonio-15-oxo-4,7,10-trioxa-14-azaoctadecan-18-yl)-4-((hydroxyimino) methyl)-pyridin-1-ium 4c

¹H NMR (400 MHz, DMSO-d₆): δ 14.25 (s, 1H), 9.02 (d, 2H, J=6.6), 8.42 (s, 1H), 8.22 (d, 2H, J=6.8), 7.93 (s, 1H), 7.84 (s, 3H), 4.58 (s, 2H), 3.48 (dd, 10H, J=11.2, 5.5), 3.37 (t, 2H, J=6.3), 3.06-3.00 (m, 2H), 2.84 (dd, 2H, J=13.5, 6.6), 2.15 (d, 4H, J=3.4), 1.81-1.74 (m, 2H), 1.58 (p, 2H, J=6.7).

¹³C NMR (101 MHz, DMSO-d₆): δ 170.74, 159.17, 158.80, 158.42, 158.05, 148.65, 145.28, 145.19, 124.15, 119.74, 116.86, 113.99, 111.11, 69.89, 69.79, 69.65, 69.61, 68.18, 67.50, 60.16, 36.91, 36.00, 31.48, 29.41, 27.30, 26.69.

### Example 4

### Preparation of Functionalized Nanodiamonds

In the reaction designated by “E” in Scheme A, 1,1′-carbonyldiimidazole (109 mg, 0.6 7 mmol) was added to a solution of ND-COOH (100 mg) in dry acetonitrile (15 mL) and stirred for 1 h. Next, a solution of the corresponding unprotected 4-hydroximinopyridinium derivate 4a, 4b, or 4c (0.56 mmol; prepared in Example 3) in acetonitrile (5 mL) was added to the reaction mixture and refluxed for 12 h. The reaction mixture was concentrated under reduced pressure to obtain the product ND-A1, ND-A2, or ND-A3 as a colored residue. After the obtained residue was filtrated, it was washed with MeOH (3×15 mL) with addition of several drops of diethyl ether and dried in vacuo.

### Example 5

### Characterization of Functionalized Nanodiamonds

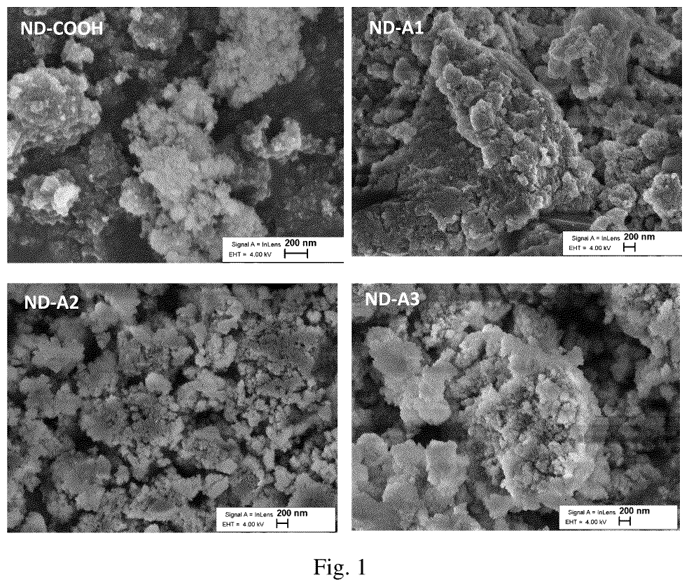

The morphologies of pristine (ND-COOH) and the functionalized nanoparticles (ND-A1, ND-A2, and ND-A3) prepared in Example 4 were characterized by scanning electron microscopy (SEM). All synthesized conjugates retained the overall texture and morphology of the starting material, albeit they visually appear slightly more agglomerated than ND-COOH (FIG. 1).

The most important characterization result, the evidence of covalent modification of the ND-COOH with functional groups, was confirmed by FT-IR and NMR spectroscopy.

### 1. Infrared Spectroscopy

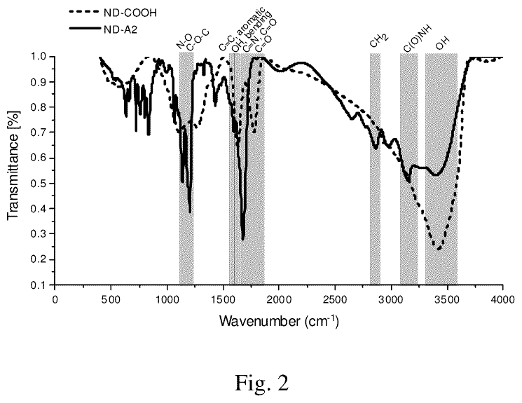

Covalent binding employed in this work is based on chemistry of —COOH groups. The carboxyl functional groups on the surface of the starting ND-COOH material were detected using the characteristic O—H stretch (3420 cm⁻¹) and bend (1628 cm⁻¹) vibrations, as well as C═O stretch band at 1775 cm⁻¹ (FIG. 2). Broad bands in the ‘fingerprint region’ (1000-1500 cm⁻¹) could be attributed to the combination of the overlapping peaks of C—O—C stretch and O—H deformational vibrations, epoxy C—O stretch and C—C stretch.

In contrast to ND-COOH, the FT-IR spectra of ND-A1, ND-A2, and ND-A3 showed new signals as the result of chemical modification. To confirm covalent attachment of molecules to nanoparticles in contrast to their adsorption, it was important to focus on the specific FT-IR bands characteristic to either new bonds formed or the existing bonds that have been broken in the attachment reactions. The coupling reaction of ND-COOH with 1,1′-carbonyldiimidazole (CDI) leads to the formation of amide bonds, which were not present in the initial material nor in the coupling agent. Accordingly, the intensity of a broad O—H stretch band decreased in the spectra of the conjugates compared to ND-COOH, and a new characteristic band of amide C(O)—N—H stretch near 3120 cm⁻¹ appeared, accompanied by a red shift of the C═O stretch peak to 1670 cm⁻¹. Additionally, the peaks of C—H stretch at 2800-3000 cm⁻¹, C═N stretch near 1650 cm⁻¹, and aromatic C—C stretch between 1600 and 1500 cm⁻¹ are seen in ND-A1, ND-A2, and ND-A3, confirming the presence of the introduced methylene, amide, and pyridinium moieties, respectively.

### 2. ¹³C MAS NMR

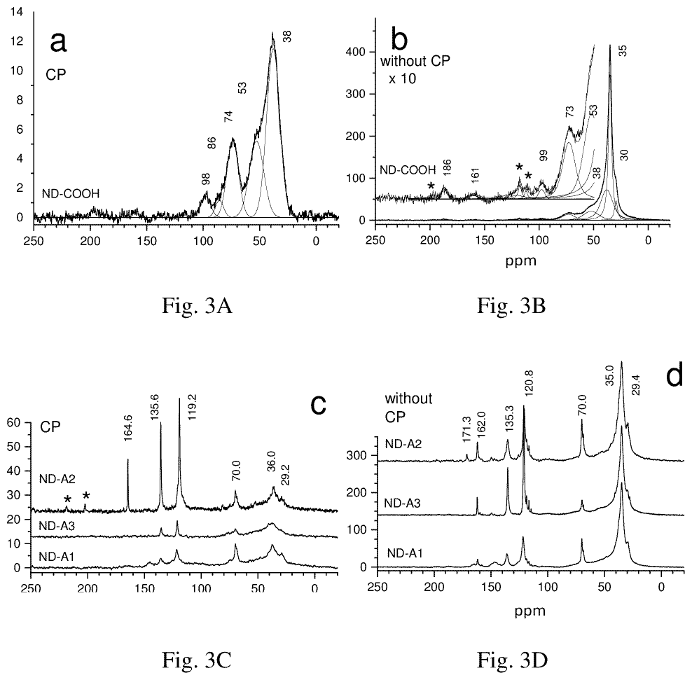

The ¹³C MAS NMR spectra of ND-COOH were obtained either via ¹H/¹³C cross polarization (CP), which highlights carbons interacting with nearby protons (FIG. 3A), or via direct excitation followed by proton decoupling, revealing different types of carbons in the sample (FIG. 3B). Naturally, in the ND conjugates the number of carbon atoms adjacent to protons is significantly smaller than the total number of carbons. Peak deconvolution using Gaussians/Lorenzians (fine, smooth (deconvoluted) lines in FIGS. 3A and 3B) shows the contributions of different components (see also Table 1), while the solid (convoluted) lines represent the convoluted spectra. Spinning sidebands (asterisks) appear at frequencies related to the sample spinning rate. FIGS. 3C and 3D display spectra of ND-A1, ND-A2, and ND-A3. Intensities exhibited in the direct excitation spectra are approximately ten times greater than those obtained with the CP pulse sequence. The spectrum recorded without ¹H/¹³C CP can be well described by eight different resonances with parameters given in Table 1. The most intense lines in the spectra with CP are ˜40 times smaller than without CP (FIG. 3B). The ratio of combined intensity of carbon atoms in the two spectra corresponds to the anticipated fraction of carbon atoms on the surface of a ca. 5 nm diameter diamond particle. Analysis of the CP spectrum shows that majority (60.8%) of carbon atoms are composed by (i) typical graphite-like carbon 2C—CH₂ at 29.5 ppm and (ii) terminal 2C—CH₂ carbon at 37.5 ppm as, e. g., in the core of adamantane, the smallest diamondoid. The resonance at 52 ppm (25.6% of surface carbons and 12.6% of all carbon atoms) could be assigned to the tertiary carbon, the peaks at 73 ppm (11.6% of carbons and 5.7% of all carbon atoms) and at 98 ppm (0.5% of all carbon atoms) occur typically in an alkyl moiety next to electron withdrawing groups. The spectrum obtained with direct excitation shows that at least half of the carbons are in the ND core sites, while the number of aromatic sp² carbons and —COOH groups is relatively small. This agrees with the results reported in NMR studies of similar ND-COOH samples.

TABLE 1
Chemical shift in the ¹³C NMR spectrum (direct excitation) of ND—COOH
with relative intensities contributed by different components.

| | | Core, | | | | | | |
|---|---|---|---|---|---|---|---|---|
| | Core | 4C—C | 2C—CH₂ | —CH₂—N— | 2C—CH—O— | 2C—C—2O | N—C═OC═N | COOH |
| Chemical | 29.5 | 34.9 | 37.5 | 51.8 | 72.8 | 98 | 161 | 187 |
| shift (ppm) | | | | | | | | |
| Relative | 4.4 | 46.4 | 29.9 | 12.6 | 5.7 | 0.5 | 0.2 | 0.3 |
| Intensity (%) | | | | | | | | |

After surface modification, new peaks belonging to the ligand moiety appear in the spectra (FIGS. 3C and 3D). These new peaks in the region of aromatic carbons at 120 ppm, 135 ppm, and 162 ppm can be assigned to the pyridine ring, the new peak at 70 ppm to the carbons in [—C—C—O—] chains, and the peak at 29 ppm to sp³ carbons of —CH₂— chains. The CP spectra of all three samples are similar, although the peaks in the aromatic region of ND-A1 and ND-A3 are weaker compared to ND-A2

### 3. Particle Size Distribution

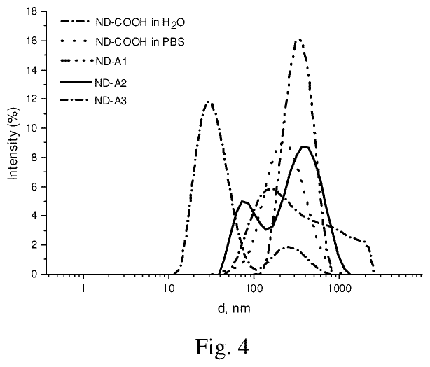

Particle size distributions of ND-COOH and oxime-functionalized ND-A1, ND-A2, and ND-A3 dispersed in demineralized water and phosphate buffer saline (PBS), modelling physiologically relevant environment, were measured by dynamic light scattering (DLS). These results are shown in FIG. 4. The suspension of ND-COOH in MilliQ water shows two populations of the aggregates in the nanoscale range, a smaller population with hydrodynamic diameter ˜30 nm and a larger fraction with a median above 200 nm. ND-COOH in PBS showed a single fraction with the aggregates of ˜250 nm diameter. The higher degree of agglomeration in PBS may be attributed to the salting-out effect commonly occurring in electrolyte solutions. In PBS suspensions, ND-A1 showed high aggregation with hydrodynamic diameter 531 nm; ND-A2 produced two overlapping populations at 164 nm and 615 nm, respectively; and ND-A3 gave a broad distribution of fractions peak at ˜250 nm.

Overall, the data show that the introduction of organic moieties on the surface of ND-COOH leads to increased aggregation in PBS and water colloids. It should be also noted that the structure and nature of ND agglomerates are still debated and that these agglomerates are most likely weakly bonded and dynamic in their nature, meaning that at any moment of time there is an equilibrium between aggregated and single ND particles in solution, which shifts towards the single particles if their equilibrium concentration is reduced.

### Example 6

### Permeability and Acetylcholinesterase Reactivation

### 1. Permeability Experiment With In Vitro BBB Model

The permeability of ND-A1 across the MDCK BBB model was studied in the double-well experiment and evaluated as the apparent permeability coefficient (Pₐₚₚ). In this model, compounds showing Pₐₚₚ>3×10⁻⁶ cm/s have high potential to enter the central nervous system and compounds with Pₐₚₚ<1×10⁻⁶ cm/s have very low chances to penetrate the BBB. The MDCK assay evaluates the ability of compounds to permeate from the donor compartment through the MDCK cell membrane into the acceptor compartment. The concentration of ND-A1 or reference compounds in both compartments was measured by UV-VIS or fluorescence spectrophotometry, with the results shown in Table 2.

TABLE 2
Permeability of ND-A1 and reference compounds
across the MDCK assay as in vitro BBB model.

| | Experimental estimation | |
|---|---|---|
| | of BBB Penetration | |
| Compound or material | Pₐₚₚ ± SD (×10⁻⁶ cm s⁻¹) | CNS (+/−)* |
| ND-A1 10 μg/mL | 14.55 ± 1.10 | CNS+ |
| ND-A1 100 μg/mL | 4.68 ± 2.46 | CNS+/− |
| Testosterone | 30.16 ± 3.99 | CNS+ |
| Donepezil | 26.80 ± 2.70 | CNS+ |
| 7-Methoxytacrine | 17.19 ± 3.56 | CNS+ |
| Sulfasalazine | 0.96 ± 1.06 | CNS− |
| Obidoxime | 1.00 ± 0.33 | CNS− |

*CNS+ stands for predicted BBB penetration; CNS− stands for no BBB penetration; CNS+/stands for uncertain result.

Remarkably, the Pₐₚₚ value for ND-A1 measured in these experiments is only about twice lower compared to either hormone testosterone or a reversible cholinesterase inhibitor donepezil, both known to be capable of crossing the BBB easily, as opposed to sulfasalazine whose Pₐₚₚ value is ˜26-30 times lower. Based on these data, the ND-A1 would be expected to permeate the BBB with an ease similar to that of 7-methoxytacrine (7-MEOTA), a reversible cholinesterase inhibitor approved for treatment of Alzheimer's disease. On the contrary, in the same experiments an established quaternary oxime antidote obidoxime demonstrates an extremely low permeability, with Pₐₚₚ value comparable to that of sulfasalazine, as expected, since BBB permeability is a known issue for charged quaternary oxime antidotes.

Interestingly, ND-A1 in a lower concentration (10 μg/mL in suspension) showed better permeability as compared to a higher concentration (100 μg/mL), which is probably due to an increased aggregation of functionalized NDs at higher concentrations or to their accumulation inside the cells. Indeed, pilot measurements using cell lysis after the MDCK permeation experiment showed that approximately 13% (in cells exposed to 10 μg/mL) and 30% (in cells exposed to 100 μg/mL) of the total amount of ND-A1 is accumulated inside the cells, confirming that NDs in higher concentrations tend to aggregate more and thus show a higher extent of intracellular entrapment.

### 2. Internalization of ND-A1 in Cells

a. MDCK Cells

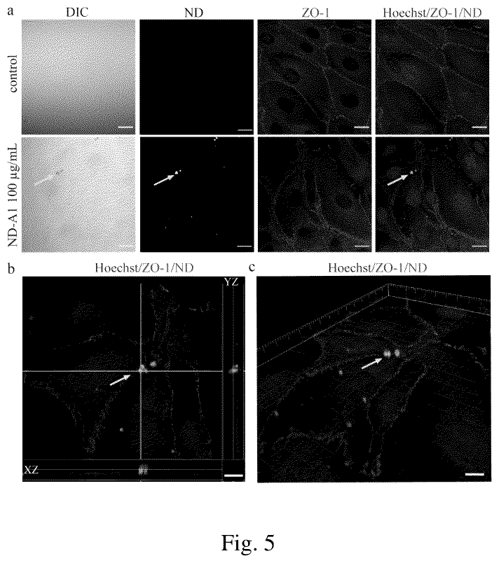

MDCK cells were used to evaluate the uptake and internalization of ND-COOH and functionalized ND-A1 in a culture that contains functionally intact, tight junctions. Zonula Occludens-1 (ZO-1) was used to stain the tight junctions that appeared intact in non-treated cells (control in FIG. 5). MDCK cells were treated with 10 μg/mL, 50 μg/mL, and 100 μg/mL of NDs for 1 h, 3 h, and 24 h. ND-A1 exhibits intrinsic green fluorescence and therefore, its visualization is possible in a convenient way, as shown in FIG. 5. ND-COOH was visualized with brightfield imaging using differential interference contrast (DIC). It was found that the internalization of ND-A1 in MDCK cells is concentration-dependent, and the nanoparticles accumulated in the cells over time. ND-A1 added at 10 μg/mL could not be visualized inside the cells up to 24 h of exposure. The MDCK cells treated with 100 μg/mL ND-A1 for 3 h showed a clear intracellular ND-A1 fluorescence (FIG. 5(a)). The tight junctions stained with ZO-1 seemed less intact and ruffled. The results are consistent with reported data on time- and concentration-dependent ND uptake via clathrin-mediated endocytosis. The 3D reconstruction with Imaris software confirmed that the ND-A1 nanoparticles successfully entered the cells as seen in the sectional and 3D view (FIGS. 5(b) and 5(c)). Disruption of tight junctions was even more noticeable at 24 h. Treatment of the cells with ND-COOH resulted in a similar concentration- and time-dependent intracellular accumulation of nanoparticles, and ZO-1 showed a similar change of its localization pattern over time as compared to the cells treated with ND-A1. Therefore, the internalization of the inventive ND-based conjugates is determined by the size and other properties of NDs and has little to no sensitivity to the attached molecules.

b. HUVEC Cells

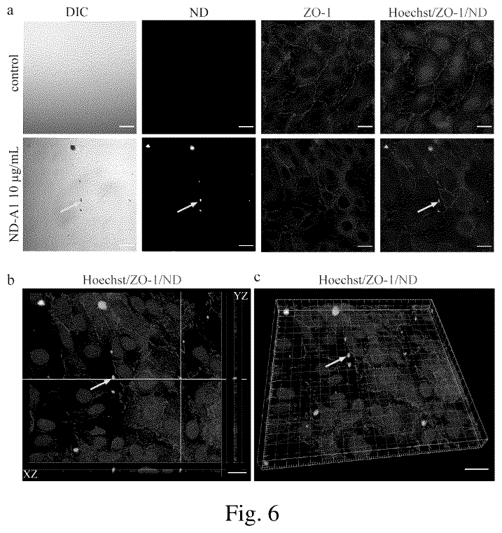

To confirm these results, another cell culture model of the human BBB, made of human umbilical endothelial cells (HUVEC), was employed. To enhance the cell-cell junctions, the cells were grown on gelatin-coated coverslips to a confluent layer and treated for 24 h with an astrocyte-conditioned medium mixed 50:50 with endothelial cell growth medium. As can be seen in FIG. 6(a), the tight junctions stained by ZO-1 are clearly visible but are less noticeable than in MDCK model, as expected. The ND-A1 had already entered the HUVEC cells at the 1 h timepoint from the solution of the lowest concentration (i.e., 10 μg/mL), as shown in FIG. 6. The HUVEC cells were reconstructed using Imaris software and rotated to better illustrate the intracellular ND-A1 particles by using sectional and 3D view (see FIGS. 6(b) and 6(c)).

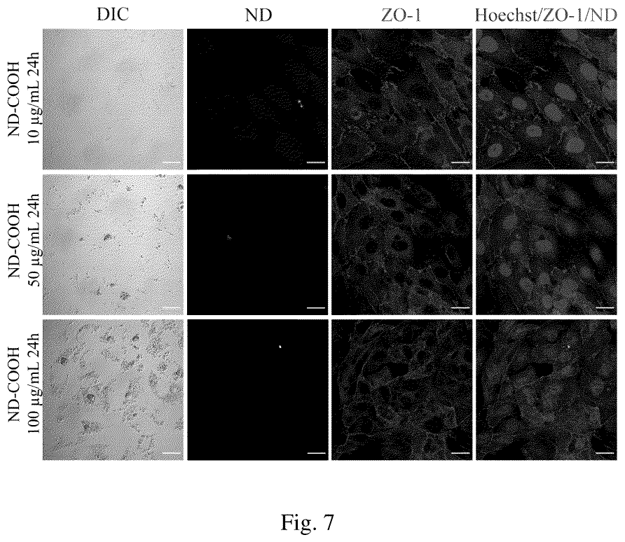

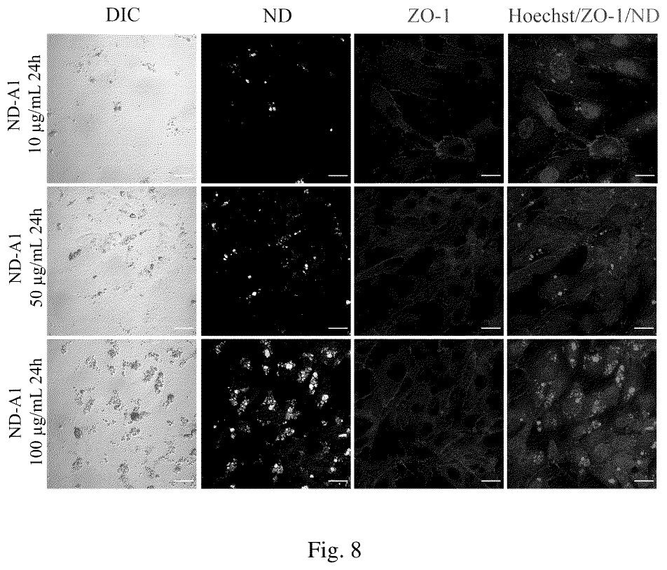

The tight junctions stained by ZO-1 were less pronounced in this cell line upon treatment with the nanoparticles, however, the difference between the ZO-1 patterns of untreated control and the cells incubated with our ND conjugates was apparent. Longer incubation periods with NDs disrupt the tight junctions between the cells considerably and in a concentration-dependent manner. The ND-A1 showed the formation of large aggregates upon entering the cell irrespective of their initial concentration (FIG. 7). However, the actin cytoskeleton of HUVEC cells seemed intact even after 24 h treatment with the highest concentration of NDs (FIG. 8). Therefore, the observations reveal that the ND-A1 nanoparticles were successfully internalized by the cells.

### 3. AChE Reactivation Using In Vitro Assays

Reactivation capacity of ND-A1, ND-A2, and ND-A3 towards human inhibited AChE was studied in comparison with obidoxime. The conjugates demonstrated notable but relatively low reactivation efficiency compared to the positive control, obidoxime, which is a pyridinium oxime with remarkable properties as an antidote in organophosphate intoxication. Surprisingly, this reactivation efficiency was not concentration dependent. Rather, an increase in concentration from 10 to 100 μg/mL changed reactivation capacity of the conjugates insignificantly or even slightly reduced it (Table 3). This could be due to an increased agglomeration of the functionalized NDs at higher concentrations, however, the use of ND conjugates in high concentrations is not practical anyway as their permeability across the BBB is lower at higher concentrations (see Table 2), and, in general, nanoparticles are intended to be used in very low concentrations as compared to larger particles. Therefore, these data further illustrate the potential of the ND-based carriers to efficiently mediate the problem of AChE reactivation in the central nervous system by facilitating the reactivator delivery across the BBB.

TABLE 3
Reactivation capacity towards AChE inhibited by nerve agents
(GB and VX) and organophosphorus pesticide paraoxon (POX).

| | GB | | VX | | POX | |
|---|---|---|---|---|---|---|
| Compound | 100 μg/mL | 10 μg/mL | 100 μg/mL | 10 μg/mL | 100 μg/mL | 10 μg/mL |
| or material | (% ± SD) | (% ± SD) | (% ± SD) | (% ± SD) | (% ± SD) | (% ± SD) |
| Obidoxime | 30.88^{a} ± 0.34 | 12.29^{b} ± 0.21 | 35.36^{a} ± 0.79 | 14.91^{a} ± 0.31 | 57.79^{b} ± 1.14 | 21.69^{b} ± 0.22 |
| ND-A1 | 0.88 ± 0.35 | 0.58 ± 0.04 | 0.80 ± 0.00 | 0.70 ± 0.70 | 1.93 ± 0.92 | 1.93 ± 0.34 |
| ND-A2 | 1.11 ± 0.41 | 1.17 ± 0.01 | 0.54 ± 0.47 | 0.71 ± 0.05 | 1.53 ± 0.38 | 2.48 ± 1.14 |
| ND-A3 | 0.56 ± 1.74 | 1.13 ± 0.52 | 1.10 ± 0.45 | 0.82 ± 0.01 | 1.89 ± 0.44 | 0.95 ± 0.02 |

^{a}100 μM
^{b}10 μM

1. A method of delivering a target compound across the blood brain barrier of a subject, said method comprising administering nanodiamonds covalently bonded to:
(a) said target compound;
(b) a linker that is covalently bonded to said target compound; or
(c) a combination of (a) and (b).

2. The method of claim 1, wherein said target compound comprises a charged molecule.

3. The method of claim 2, wherein said target compound comprises a positively charged molecule.

4. The method of claim 3, wherein said target compound comprises a quaternary oxime.

5. The method of claim 1, wherein said target compound is chosen from pralidoxime, asoxime, obidoxime, trimedoxime, methoxime, or mixtures thereof.

6. The method of claim 1, wherein said nanodiamonds comprise detonation nanodiamonds.

7. The method of claim 1, wherein said nanodiamonds comprise primary particles having an average particle size of about 3 nm to about 6 nm.

8. The method of claim 1, wherein said nanodiamonds covalently bonded to (a), (b), or (c) form aggregates having an average hydrodynamic diameter of about 150 nm to about 620 nm.

9. The method of claim 1, wherein said nanodiamonds comprises surface —COOH groups.

10. The method of claim 1, wherein said linker is biocompatible.

11. The method of claim 1, wherein said linker comprises 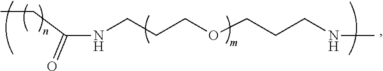
where m is 1 to 5, and n is 1 to 5.

12. The method of claim 1, where said subject has been exposed to organophosphorus poisoning.

13. The method of claim 12, wherein said organophosphorus poisoning was the result of exposure to a compound chosen from nerve agents, toxins, pesticides, simulants for pesticides, herbicide, or mixtures thereof.

14. The method of claim 13, wherein said compound is chosen from triphenyl phosphate, methyl paraoxon, paraoxon, chlorpyrifos, chlorpyrifos oxon, malaoxon, O,O-diethyl O-(4-nitrophenyl) phosphorothioate, O,O-Dimethyl O-(3-methyl-4-nitrophenyl) phosphorothioate, isopropyl methylphosphonofluoridate, (RS)-ethyl N,N-dimethylphosphoramidocyanidate, cyclohexyl methylphosphonofluoridate, (O-ethyl-S-[2 (diisopropylamino)ethyl]methylphosphonothioate), O-butyl-S-[2-(diethylamino)ethyl]methylphosphonothioate, S-[2-(diethylamino)ethyl]-O-(2-methylpropyl) methylphosphonothioate, 3,3-dimethylbutan-2-yl methylphosphonofluoridate, 2-(dimethylamino)ethyl N,N-dimethylphosphoramidofluoridate, methyl-(1-(diethylamino)ethylidene)phosphonamidofluoridate, methoxy-(1-(diethylamino)ethylidene)phosphoramidofluoridate, ethyl N-[(1E)-1-(diethylamino)-ethylidene]-phosphoramidofluoridate, or mixtures thereof.

15. The method of claim 1, wherein said nanodiamonds covalently bonded to (a), (b), or (c) at a concentration of about 10 μg/mL have an apparent permeability coefficient according to the MDCK blood-brain barrier model of 3×10⁻⁶ cm/s or greater.

16. The method of claim 1, wherein at least one of the following is true:
(a) MDCK cells treated with said nanodiamonds covalently bonded to (a), (b), or (c) at a concentration of about 50 μg/mL internalize said nanodiamonds bonded to (a), (b), or (c) about 24 hours after treatment, as visualized by fluorescence microscopy at a wavelength of about 254 nm;
(b) MDCK cells treated with said nanodiamonds covalently bonded to (a), (b), or (c) at a concentration of about 100 μg/mL internalize said nanodiamonds bonded to (a), (b), or (c) about 3 hours after treatment, as visualized by fluorescence microscopy at a wavelength of about 254 nm; or
(c) HUVEC cells treated with said nanodiamonds covalently bonded to (a), (b), or (c) at a concentration of about 10 μg/mL internalize said nanodiamonds bonded to (a), (b), or (c) about 1 hour after treatment, as visualized by fluorescence microscopy at a wavelength of about 254 nm.

17. The method of claim 1, wherein at least one of the following is true:
(a) said nanodiamonds covalently bonded to (a), (b), or (c) at a concentration of about 10 μg/mL have an AChE reactivation potency R of about 0.55% or greater against isopropyl methylphosphonofluoridate (GB);
(b) said nanodiamonds covalently bonded to (a), (b), or (c) at a concentration of about 10 μg/mL have an AChE reactivation potency R of about 0.7% or greater against O-ethyl S-diisopropylaminomethyl methylphosphonothiolate (VX); or
(b) said nanodiamonds covalently bonded to (a), (b), or (c) at a concentration of about 10 μg/mL have an AChE reactivation potency R of about 0.95% or greater against O,O-diethyl O-(4-nitrophenyl) phosphate (POX).

18. A nanodiamond having a surface group that is covalently bonded to a positively charged molecule chosen from pralidoxime, asoxime, obidoxime, trimedoxime, methoxime, or mixtures thereof.

19. The nanodiamond of claim 18, further comprising a linker that is covalently bonded with both said surface group and said positively charged molecule.

20. The nanodiamond of claim 19, wherein said linker comprises 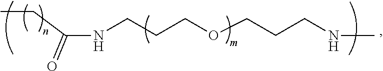
where m is 1 to 5, and n is 1 to 5.
